(19) United States
(12) Patent Application Publication
(10) Pub. No.: US 2006/0281147 A1
(43) Pub. Date: Dec. 14, 2006

(54) Thrombin-like recombinant baxtroxobin expressed by pichia sp.and production method thereof

(75) Inventors: Weon Kyoo You, Seoul (KR); Won Seok Choi, Incheon (KR); You Seok Koh, Seoul (KR); Seung Woo Kang, Seoul (KR); Kwang Hoe Chung, Seoul (KR)

Correspondence Address: THE RAFFERTY PATENT LAW FIRM, 5641 BURKE CENTRE PKWY, SUITE 100, BURKE, VA, 22015-1731, US

(73) Assignee: BIOBUD CO., LTD., Seoul (KR)

(21) Appl. No.: 10/595,576

(22) PCT Filed: Oct. 31, 2003
(86) PCT No.: PCT/KR03/02318

§ 371 Date: Apr. 28, 2006

(30) Foreign Application Priority Data
Oct. 31, 2003 (WO) PCT/KR03/02318

(51) Int. Cl. C12P 21/06; C12N 9/74; C07H 21/04; C12N 15/74; C12N 1/18

(52) U.S. Cl. 435/69.1; 536/23.2; 435/483; 435/254.23; 435/214

(57) ABSTRACT

A recombinant thrombin-like enzyme batroxobin expressed by yeast, a production method thereof, and use of the batroxobin as a hemostatic agent or antithrombotic agent is disclosed. In order to express recombinant thrombin-like enzyme, an expression vector is prepared by inserting cDNA encoding the enzyme, the transformed cell is prepared by introducing the expression vector into the cell, the transformed cell then is incubated and the recombinant thrombin-like enzyme is obtained from the cell. The recombinant thrombin-like enzyme may be usefully used as a hemostatic agent because the enzyme effectively decrease bleeding time and blood coagulation time and does not affect various blood coagulation factors. The recombinant thrombin-like enzyme is also useful for a hemostatic agent or antithrombotic agent comprising the recombinant thrombin-like enzyme as an active component

### TECHNICAL FIELD

The present invention relates to recombinant thrombin-like enzyme, batroxobin, expressed from yeast and purification method thereof. More in detail, the present invention relates to recombinant thrombin-like enzyme (recombinant batroxobin) expressed from yeast (Pichia pastoris) and method of production and purification by using thrombin-like enzyme (batroxobin) isolated from the venom of Latin venomous snake (Bothrops atrox moojeni) with gene recombination technique.

### BACKGROUND ART

Generally, venom effect on blood coagulation cascade and fibrinolytic pathway of mammalian including human has been investigated for a long time and several effective agents have been isolated and characterized. Various components included in venom are known to affect fibrin-clotting, platelet aggregation and so on directly or indirectly, thus to be used as pro-coagulant or anti-coagulant (See Meaume, J. Toxicon, U.S. Pat. No. 4, 2558, 1966 Matsui et al., Biochim. Biophys. Acta., 1477, 146-156, 2000). Some of the components are already characterized and broadly used for diagnosis and therapy of thrombosis. In particular, the study about thrombin-like enzyme converting fibrinogen into fibrin by cleavage of fibrinopeptide has been performed actively and over 20 proteins have been reported, and cDNA of some was characterized. The thrombin-like enzyme initially hydrolyze fibrinopeptide A of fibrinogen molecule to make unstable fibrin clot (des-A-fibrin) unlike thrombin, mammalian native blood coagulation protein, but the unstable fibrin clot is rapidly degraded by in vivo fibrinolysis system over time to eventually decrease blood fibrinogen level (See Pirkle, H., and Stocker, K. Thromb. Haemost. 65, 444-450, 1991 Marsh, N.A., Blood Coagul. Fibrinolysis, 339-410, 5, 1994).

Therefore the thrombin-like enzyme is used in clinic field as hemostatic agent or therapeutic and preventing agent for thrombosis by using these both-sided characteristics of enzyme. This enzyme don't also have an influence on other blood coagulation factors and activation of platelet, with which merit it shows effective hemostatic activity to intravenously or intramuscularly inject the small amount of the enzyme (2 NIH unit/60 kg) 1-2 hours before surgery. On the other hand, it is possible to reduce blood fibrinogen level without side effects such as bleeding, that can be happen when using thrombolytic enzyme, by controlling dose and administration time of enzyme. The release of des-A-fibrin and FDP (fibrinogen degradation products) formed during the above process stimulate hemoendothelial cell to induce the production of plasminogen activator. The enzyme is used as therapeutic and preventing agent for thrombosis because the enzyme can inhibit thrombin activity (See Schumacheret al., Thromb. Res. 81, 187-194, 1996; Bell W. R. Jr., Drugs, 54, 18-30, 1997). Recently, this fibrinogen reduction effect of the thrombin-like enzyme is reported to be effective on therapy of heparin-induced thrombocytopenia (thrombosis) or acute ischemic stroke caused by administration of heparin (See Dempfle et al. Blood, 96, 2793-2802, 2000).

The clinically used all thrombin-like enzymes are native proteins isolated and purified from venom. Batroxobin isolated from venom of Latin venomous snake Bothrops atrox moojeni is commercially available from Italian Solco Basle Ltd. Company and Swiss Pentapharm Company and is sold as trade names like reptilase (for hemostasis), defibrase (for thrombolysis), reptilase-reagent (for diagnosis reagent). Botropase (for hemostasis, Italian Ravizza Company) isolated from venom of Latin venomous snake Bothrops jararaca, Malayan pit viper and Ancrod (American Knoll Pharmaceutical Company) isolated from venom of Calloselasma rhodostoma are also commercially available. It is reported that 20 thrombin-like enzymes except the above clinically used proteins were isolated from various venoms and characterized. cDNA of some were also characterized. Because of high clinical value, the study about expression and purification of recombinant protein has been performed until now, but any successful case to largely express recombinant thrombin-like enzyme is not reported yet. The investigation to express recombinant thrombin-like enzyme from E. coli has largely performed, but most recombinant thrombin-like proteins are expressed as a form of insoluble inclusion body and need refolding process to be changed to soluble active protein (See Yang et al., Biotechnol. Lett., 25, 101-104, 2003; Fanet al., Biochem. Mol. Biol. Int. 47, 217-225, 1999; and Maeda et al., J. Biochem., 632-637, 109, 1991). Because thrombin-like enzyme has complex tertiary structure having many number (6) of disulfide bond compared to molecular weight (about 30 kDa), it is difficult to establish refolding condition. Because of this difficulty, there is up to date no successful result to refold recombinant thrombin-like enzyme expressed from E. coli and make the thrombin-like enzyme have comparable activity to native enzyme.

### DISCLOSURE OF INVENTION

The present invention is made to solve the above problem and satisfy the above need. Purpose of present invention relates to recombinant thrombin-like enzyme isolated from venom of Latin snake (Bothrops atrox moojeni) and expressed from yeast, and mass production and preparation method of the protein.

Another purpose of the present invention is to provide an expression vector of the above-described yeast-expressed recombinant thrombin-like enzyme.

Yet other purpose of the present invention is to provide transformant transformed with the above-described expression vector.

Yet other purpose of the present invention is to provide fermentation method and purification process for mass production of the above-described recombinant thrombin-like enzyme.

Yet other purpose of the present invention is to provide a therapeutic and preventing agent for thrombosis comprising the above-described recombinant thrombin-like enzyme as effective agent.

To achieve the above purpose, the present invention yeast expression vector pPIC-rBAT expressing recombinant thrombin-like enzyme.

Preferably, the recombinant thrombin-like enzyme is batroxobin protein listed in Sequence No: 1.

The present invention also provides yeast (Pichia pastoris) GS115 transformed with the expression vector pPIC-rBAT.

In addition, the present invention provides preparation method of thrombin-like enzyme, comprising culture and fermentation of the transformed microorganism and obtaining recombinant thrombin-like enzyme from the microorganism.

In the present invention, the preferable method is to incubate the transformed microorganism in the medium of pH 5.0-7.0 at 20-40° C. for 12-14 hours and then incubate the cells obtained by centrifugation of culture in the medium of pH 5.0-7.0 containing 0.5-1.5% (v/v) methanol at 20-40° C. for 72-120 hours.

The present invention also provides preparation method of recombinant thrombin-like enzyme comprising a) process to gain active fraction of thrombin-like enzyme by using hydrophobic chromatography from the expressed culture fluid; and b) process to purify recombinant thrombin-like enzyme by applying the active fraction to affinity chromatography.

### BRIEF DESCRIPTION OF THE DRAWINGS

These and other features, aspects, and advantages of preferred embodiments of the present invention will be more fully described in the following detailed description, taken accompanying drawings. In the drawings:

Fig. 1a shows the activity of recombinant thrombin-like enzyme compared to the activities of native thrombin-like enzyme and native thrombin using synthetic chromogenic substrate.

Fig. 1b shows the activity of recombinant thrombin-like enzyme compared to native thrombin-like enzyme by measuring plasma clotting time.

FIG. 2 is the gene map of the expression vector pPIC-rBAT of recombinant thrombin-like enzyme.

FIG. 3 is the result of SDS-PAGE analysis on the fractions obtained during expression and purification of recombinant thrombin-like enzyme. Lane 1 is the protein fraction precipitate of the incubation medium of yeast expressing recombinant thrombin-like enzyme, lane 2 is the recombinant thrombin-like enzyme fraction isolated from the medium by using hydrophobic column chromatography, and lane 3 is the recombinant thrombin-like enzyme finally purified from the fraction of lane 2 by using affinity column chromatography. Lane 4 is the result of SDS-PAGE analysis on the commercially available native thrombin-like enzyme (Pentapharm, Basel, Switzerland).

FIG. 4a shows the result of hydrophobic column chromatography that was the first purification step of recombinant thrombin-like enzyme and the fraction indicated as the bold line has the strong activity of thrombin-like enzyme.

FIG. 4b shows the result of affinity column chromatography that was the second purification step of recombinant thrombin-like enzyme and the fraction indicated as bold line has the activity of thrombin-like enzyme.

Fig. 5a shows the fibrin clot formed by the recombinant thrombin-like enzyme.

Fig. 5b shows the fibrin clotting activity of recombinant thrombin-like enzyme by using reverse zymography.

FIG. 6a shows the comparison of the bleeding time reduction caused by recombinant thrombin-like enzyme and native protein in animal experimental model. Recombinant thrombin-like enzyme and native thrombin-like enzyme (1 NIH unit/kg, respectively) were injected on the tail vein of rats. After 1 and half hours, the tail of rats was transected at 5 mm from the tip and the time to stop bleeding was measured with the tail in PBS (phosphate buffered saline). The control was injected with only PBS.

FIG. 6b shows the comparison of the whole blood coagulation time reduction caused by recombinant thrombin-like enzyme and native protein in animal experimental model. Recombinant thrombin-like enzyme and native thrombin-like enzyme (2 NIH unit/kg, respectively) were injected on the tail vein of rats. After 1 hour and 4 hours, respectively, the blood was collected and the whole blood coagulation time was measured. The control was injected with only PBS.

FIG. 6c shows the changes of PT, APTT and TT caused by recombinant thrombin-like enzyme and native protein in animal experimental model. Recombinant thrombin-like enzyme and native thrombin-like enzyme (0.1 NIH unit/kg, respectively) were injected on the tail vein of rats. After 2 hours, the blood was collected and the plasma was isolated. The PT, APTT and TT for each plasma were measured by using automatic blood clotting tester. The control animals were injected with only PBS.

FIG. 7 shows the comparison of the stability of recombinant thrombin-like enzyme and native protein according to the pH.

### BEST MODES FOR CARRYING OUT THE INVENTION

Hereinafter, preferred embodiments of the present invention will be described in detail with reference to the accompanying drawings.

The present inventors recombinant thrombin-like enzyme that can be expressed and secreted from yeast by using gene recombination technique with cDNA of clinically used thrombin-like enzyme. The successful result to mass-produce recombinant thrombin-like enzyme in yeast expression is not reported yet. The thrombin-like enzyme of the present invention has advantage that doesn't need refolding process to change inclusion body to active recombinant protein like the protein expressed from E. coli because the yeast-expressed recombinant thrombin-like protein is made to be secreted outside yeast cell. In addition, the thrombin-like enzyme of the present invention is believed to have advantage that it can simplify purification process and increase yield over the process to make native thrombin-like enzyme from venom because the expressed recombinant thrombin-like enzyme is secreted outside yeast cell.

The present inventors also developed the technique to mass-produce recombinant thrombin-like enzyme through yeast fed-batch fermentation using gene recombination technique. The produced enzyme is secreted outside yeast cell and purified thrombin-like enzyme can be made through some purification process. The SDS-PAGE analysis showed that the recombinant thrombin-like enzyme of the present invention has high purity than native thrombin-like enzyme. Several identification tests for activity showed that the recombinant thrombin-like enzyme of the present invention has 1.5 times greater specific activity. Moreover, the pre-clinical toxicity test demonstrated that the yeast-expressed recombinant thrombin-like enzyme has lower toxicity than native thrombin-like enzyme, and pharmacological efficacy test using animal experimental model showed that the efficacy of thrombin-like enzyme of the present invention is stronger than native protein. Stability test also showed that recombinant thrombin-like enzyme of the present invention has good stability over broad pH compared to the native protein. Therefore, yeast-expressed recombinant thrombin-like enzyme has many advantages over the existing native enzyme when is clinically used.

To express and purify recombinant protein batroxobin, which is known to have the strongest activity among thrombin-like enzymes, cDNA (GeneBank Accession No. AF056033) of thrombin-like enzyme (salmobin) isolated from Korea venomous snake (Gloydius halys) is used as a template and is transformed to cDNA encoding batroxobin with gene recombination technique. Vector expressing recombinant thrombin-like enzyme is constructed by using the cDNA of batroxobin and the transformant is made by using the vector. As a result, recombinant thrombin-like enzyme is made from the transformant.

Hereinafter, recombinant thrombin-like enzyme and preparation method of the present method is described in detail.

The cDNA base sequence of salmobin, thrombin-like enzyme found in cDNA library made from venom secreting gland of Korea venomous snake, is compared with cDNA base sequence of batroxobin and is transformed to cDNA encoding batroxobin with gene recombination technique. To express from yeast and purify recombinant thrombin-like enzyme (recombinant batroxobin), base sequence of restriction enzyme (XhoI) and base sequence encoding amino acid sequence that can be cleaved with protease KEX2 are inserted in the terminal of cDNA of the transformed batroxobin. Then expression vector pPIC-rBAT is constructed by inserting the cDNA in the left side of XhoI-EcoRI of the c-terminal of a-factor secretion signal protein of pPIC9 (Invitrogen, USA), 8.0 kbp yeast expression vector. The constructed expression vector is introduced into yeast such as Pichia genus, Hansenulla genus, Saccharomyces genus and so on to make transformant. Preferably, the yeast is Pichia pastoris species like Pichia pastoris GS115, SMD 1168, KM71 and so on.

The transformant is made by introducing the vector pPIC-rBAT into Pichia pastoris GS115. The transformant is named GSrBAT.

The preparation method of recombinant thrombin-like enzyme comprises culture of the transformant and purification of recombinant thrombin-like enzyme from the culture: at this point, the culture method of the transformant is to inoculate the transformant on minimal glycerol medium and incubate the transformant until O.D600 is 1.0, and to obtain cells isolated culture fluid by centrifugation, and to suspend the cells in the minimal methanol medium, and incubate in fed-batch fermentation. The used minimal glycerol medium contains 0.5-1.5% yeast extract or pepton as nitrogen source, 0.5-2.5% glycerol, dextrose or glucose as carbon source, and minimum amount of biotin. The pH of minimal glycerol medium is pH 5.0-7.0, preferably 5.5-6.5, optimally 6.0. The minimal methanol medium is minimal glycerol medium containing 0.1-1.0% (v/v) methanol based on the volume of medium as carbon source, preferably 0.3-0.8% (v/v) methanol, most preferably 0.5% (v/v) methanol. The culture condition is 20-40° C., preferably 25-35° C., most preferably 30° C. and culture time is 12-14 hours, preferably 16-20 hours, most preferably 18 hours.

Purification process of recombinant thrombin-like enzyme is to apply the culture fluid containing the incubated transformant to hydrophobic chromatography and affinity chromatography to get pure recombinant thrombin-like enzyme. The preferable resin packed in hydrophobic column is phenyl-Sepharose, butyl-Sepharose, octyl-Sepharose. The preferable mobile phase is 0.5-2M ammonium sulfate solution. The preferable affinity column is heparin-Sepharose and benzamidnine-Sepharose column and the preferable mobile phase is Tris-HCI buffered solution containing NaCl.

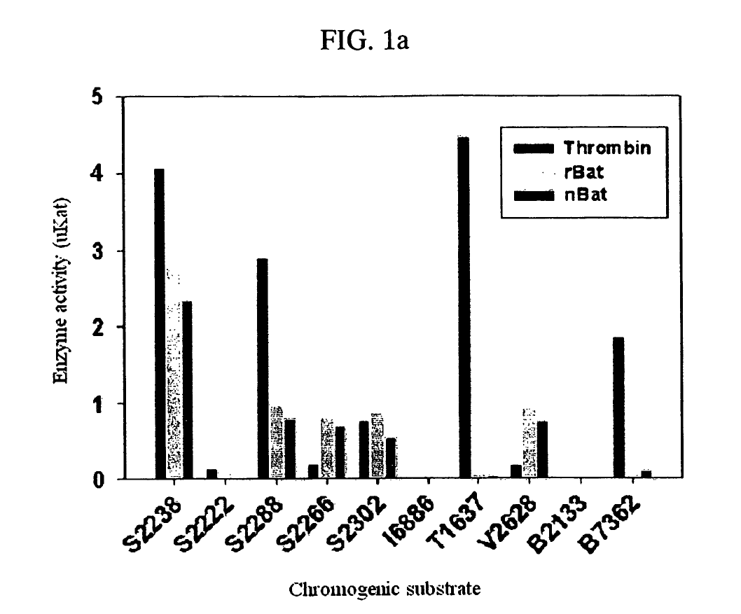

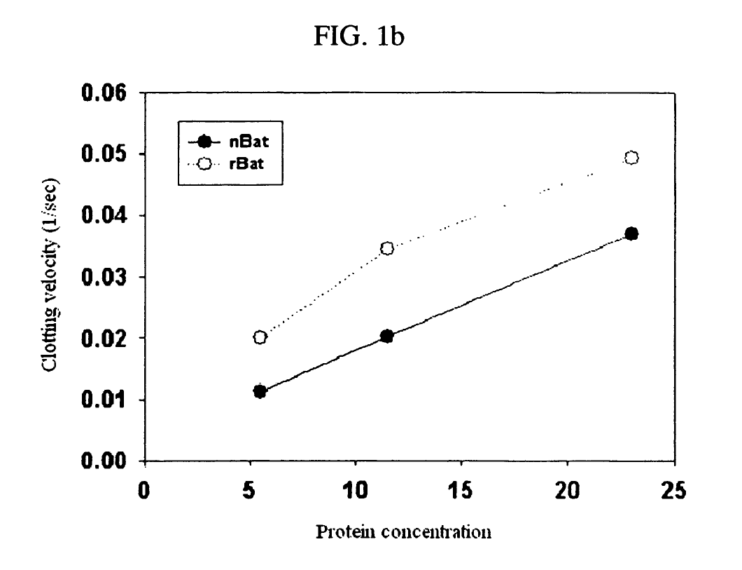

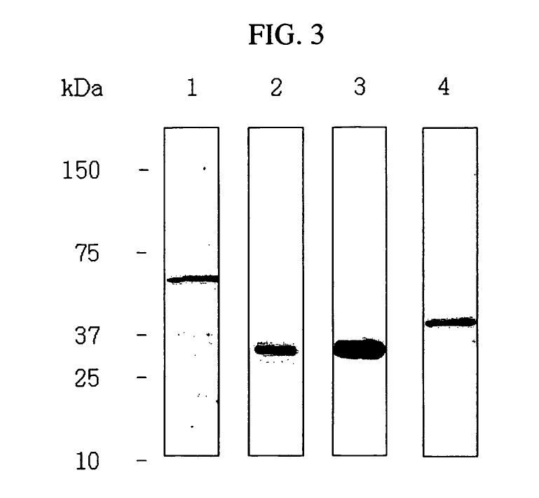

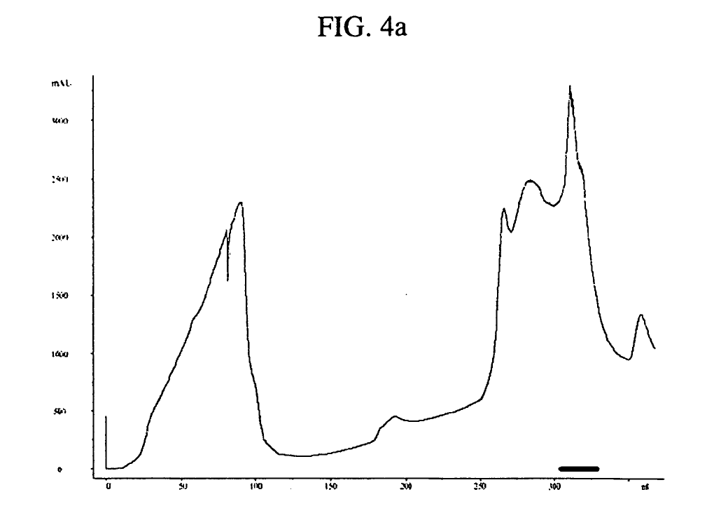

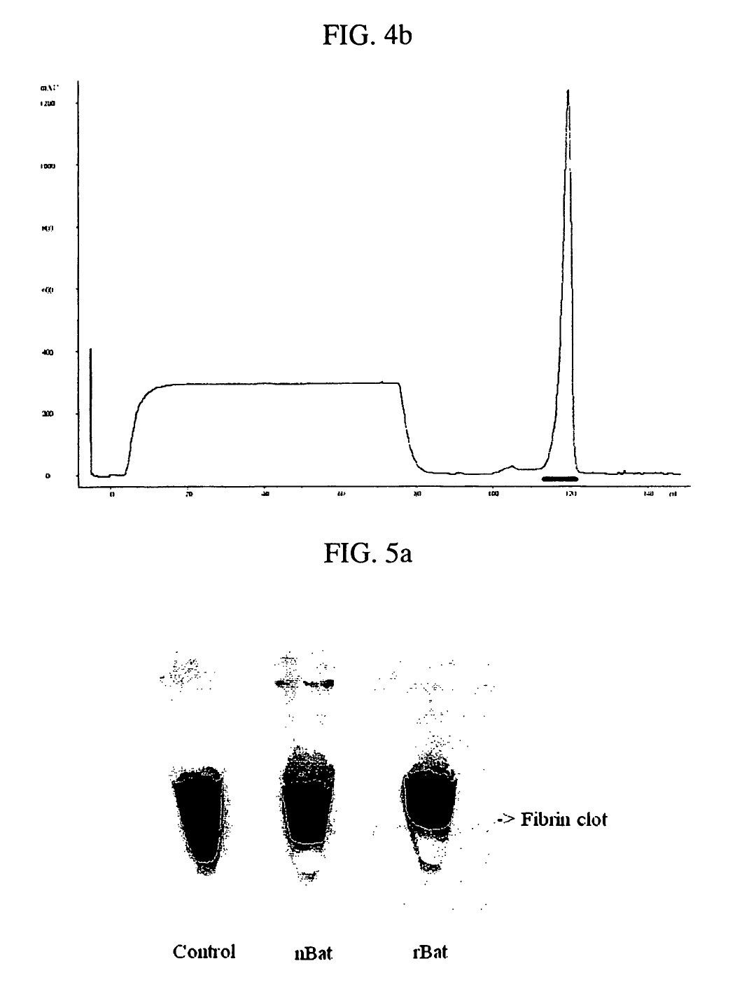

Comparison test of synthetic chromogenic substrate catalytic activity and fibrinogen clotting activity between the recombinant thrombin-like enzyme and the commercially available native batroxobin of Swiss Pentapharm Company shows that the specific activity of recombinant thrombin-like enzyme of the present invention is 1.5 times stronger (FIG. 1). In addition, SDS-PAGE analysis of the finally purified recombinant thrombin-like enzyme and native thrombin-like enzyme demonstrates that the purity of yeast-expressed recombinant thrombin-like enzyme of the present invention is higher than the native (FIG. 3). The present invented purification process of recombinant thrombin-like enzyme has advantage to use only simple two columns over the complex purification process of native enzyme, which is expected to increase a yield of recombinant protein (FIG. 4).

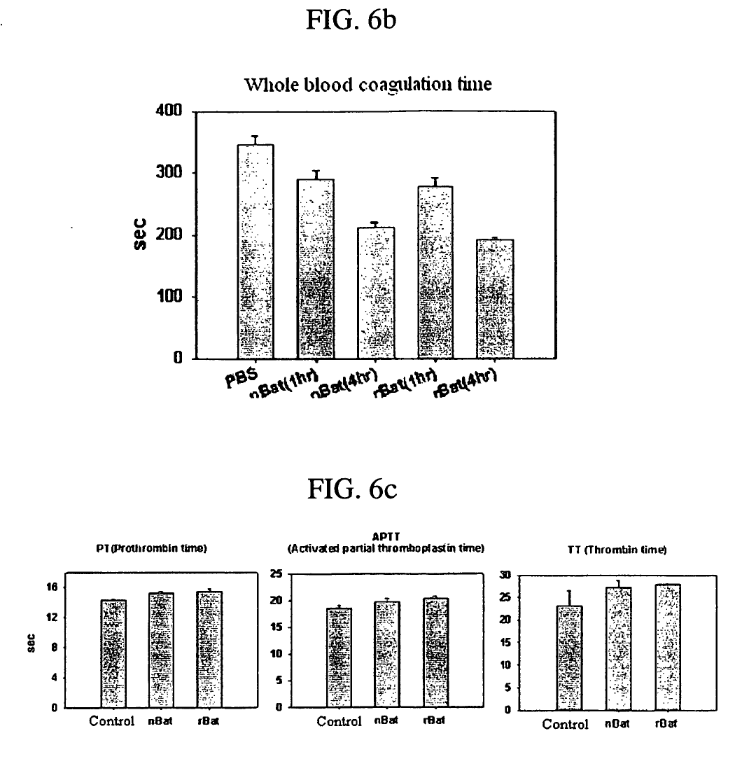

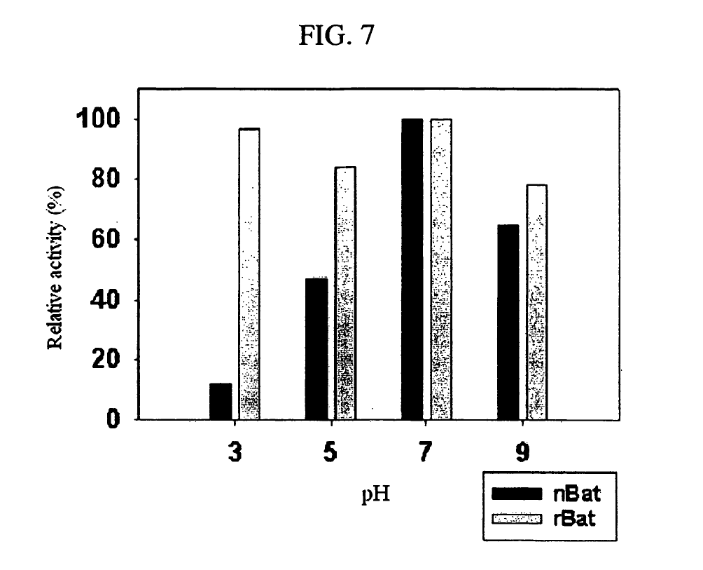

At the same time, to investigate whether recombinant thrombin-like enzyme has hemostatic activity in actual animal experimental model, the hemostatic effect and effects on blood coagulation factors of recombinant thrombin-like enzyme and native protein in rat model was measured by using ACL automated blood coagulation instrument. Results showed that recombinant thrombin-like enzyme has hemostatic activity such as effective reduction of bleeding time and whole blood coagulation time, and the activity is stronger than the native (See FIG. 6a and 6b). Both recombinant thrombin-like enzyme and native thrombin-like enzyme do not have any significant influence on other blood coagulation factors (FIG. 6c). In vivo and in vitro test showed that the specific activity of recombinant thrombin-like enzyme is greater than the native, which is expected to be related with the stability of the enzyme. Thus the stability of recombinant thrombin-like enzyme and native recombinant thrombin-like enzyme was investigated. The stability test using synthetic chromogenic substrate demonstrated that the stability of recombinant thrombin-like enzyme is much better than the native protein in several pH conditions (FIG. 7)

Hereinafter, the present invention is described in considerable detail by using examples. The following examples are offered only to illustrate the present invention. It is apparent to those skilled in the art that these examples are not intended to limit the scope of the invention.

### EXAMPLE 1

### Cloning of Recombinant Thrombin-like Enzyme

Among thrombin-like enzymes, the cloning of cDNA for recombinant protein expression of batroxobin that is currently used in clinics and has the strong hemostatic activity was performed by using cDNA of new thrombin-like enzyme (salmobin) isolated from Korea snake (Gloydius halys). The amino acid sequence of new thrombin-like enzyme (salmobin) isolated from Korea snake shows high homology to batroxobin. The oligoprimer for deformity was synthesized and the oligoprimer was composed of the moiety having the base sequence identically existing in both proteins and the moiety having the unique base sequence existing in only batroxobin. The cDNA of batroxobin, thrombin-like enzyme derived from Latin snake venom, was cloned by repeatedly doing polymerase chain reaction (hereinafter, “PCR”) with the oligoprimer.

### EXAMPLE 2

### Construction of Expression Vector for Recombinant Thrombin-like Enzyme

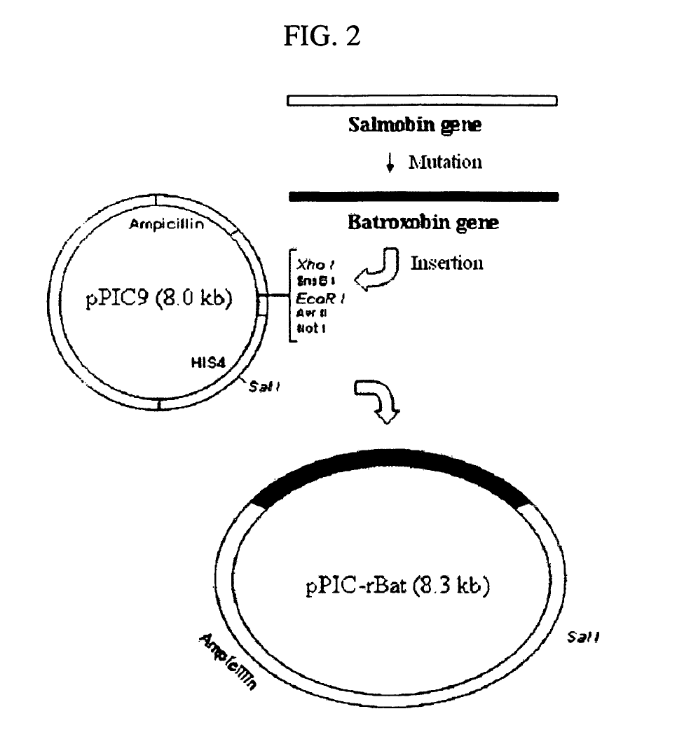

PCR was performed with the N-terminal primer 5′-CTCGAGAAAAGAGTCATTGGAGGTGATG-3′(Sequence NO: 2) containing restriction enzyme Xhol base sequence and base sequence encoding amino acid sequence which could be cleaved by proteolytic enzyme KEX2, the C-terminal primer (Sequence NO: 3) 5′-TTCACGGGCAAGTCGCAGTTTTATTTTCctGCAA tAATcgTC-3′containing two translation stop codons and restriction enzyme BamHI site, and the plasmid having the cDNA of the thrombin-like enzyme constructed in the above Example 1 as the template, using the Robocycler™ (Stratagen, USA). PCR cycle was denaturation (94° C., 90 seconds), annealing of the template and the primer (52° C., 60 seconds), and primer polymerization (72° C., 90 seconds) and 30 cycles were carried out. Recombinant plasmid (pGEM-rBAT) was constructed by transferring the above-amplified 709 bp DNA fragment into the cloning plasmid pGEM-Teasy (3.015 kbp, Promega, USA) for PCR products and using T4 DNA ligase, and then the plasmid was transformed into E. coli XL1 Blue to make the transformant. The transformant was incubated in the Luria Botani (LB) plate medium containing ampicillin 50 μg/ml and then the transformed E. coli colonies showing white color were selected. The plasmid (pGEM-rBAT) was isolated from the colonies and was analyzed using restriction enzyme map analysis and DNA sequence analysis (Genotech, Daejeon, Korea). The results showed that the 709 bp fragment amplified with PCR was the cDNA of recombinant thrombin-like enzyme for yeast expression. The DNA fragment obtained by cleaving the electrically isolated plasmid pGEM-rBAT with XhoI and BamHI was inserted into the C-terminal site of α-factor secretion signal protein of yeast expression vector pPIC9 (8.0 kbp), which constructed yeast expression vector pPIC-rBAT (pPIC9 \ddot{r}ecombinant Batroxobin: 8.7 kbp) of the recombinant thrombin-like enzyme (See FIG. 2).

### EXAMPLE 3

### Construction of pPIC-rBAT Transformant

The linear DNA was gained by cleaving the above constructed pPIC-rBAT with SalI, and then the TE buffered solution containing the linear DNA (0.5, μg/μl.) and Pichia pastoris (GS115 strain, Invitrogen) competent cell 80μl were mixed. The transformation was performed under the voltage condition of 1.5 KV using Electroporator(Bio-Rad Gene Pulser, U.S.A.). After that, the transformed strain was inoculated on the histidine-defective agarose plate medium and was incubated at 30° C. for 3 days. After incubation, the cultured colony was chosen and was inoculated on minimal glycerol medium (100 mM Sodium Phosphate pH 6.0, Yeast Nitrogen Base 1.34%, biotin 4×10⁻⁵%, and glycerol 1%) 1 L followed by incubation at 30° C. until the concentration of cell became O.D600 1.0. The medium-free cell was obtained by centrifuging the incubated medium at 3,000×g and the obtained cells were suspended in minimal methanol medium (100 mM Sodium Phosphate pH 6.0, Yeast Nitrogen Base 1.34%, biotin 4×10⁻⁵%, and methanol 0.5%), and incubated at 30° C. to induce the expression of recombinant thrombin-like enzyme. Then, 0.5% methanol was added with the interval of 24 hours and the cells was incubated for 96 hours. As a result of incubation, the accumulation of recombinant thrombin-like enzyme in the medium was identified. The transformed strain was named “GSrBAT”.

### EXAMPLE 4

### Expression and Purification of Recombinant Thrombin-like Enzyme

The culture fluid was gained by centrifuging the above yeast culture medium at 5,000×g, and was loaded to phenyl-Sepharose column (1.3×20 cm, Amerchambioscience, U.S.A.) equilibrated with 2.5M ammonium sulfate solution followed by elution with a linear gradient of 2.5-0M ammonium sulfate at the flow rate of 0.5 ml/min to get recombinant thrombin-like enzyme active fraction (See FIG. 4a). The activity of recombinant thrombin-like enzyme was measured in the same way as example 1. After that, the obtained active fractions were pooled and dialyzed against 20 mM Tris-HCI buffered solution (pH 7.5) for 8 hours (3 times), and was loaded to affinity column (heparin-Sepharose, 1×5 cm) equilibrated with the same buffered solution followed by elution with a linear gradient of 0-1M the same buffered solution containing 1M NaCl at the flow rate of 1 ml/min to get purely isolated recombinant thrombin-like enzyme (FIG. 4b). The yield was 7 mg per 1 L culture fluid.

To analyze the amino acid sequence of recombinant thrombin-like enzyme, the purified recombinant thrombin-like enzyme was electrophoresed under reducing condition, and electroblotted onto PVDF (Bio-Rad, U.S.A.) membrane and the N-terminal amino acid sequence was analyzed using automatic amino acid sequence analyzer. The result showed that the sequence was the sequence of VIGGDECDIN, which means that recombinant thrombin-like enzyme was correctly expressed in the yeast.

### EXAMPLE 5

### Comparison of Activity of Recombinant Thrombin-like Enzyme and Native Thrombin-like Enzyme

The catalytic activity of chromogenic substrate synthesized to compare the activity of recombinant thrombin-like enzyme and native thrombin-like enzyme was measured, and the changes of human plasma clotting time was measured by using ACL automated blood coagulation instrument according to sample concentration. The catalysis of the synthetic chromogenic substrate caused by contacting recombinant thrombin-like enzyme or native thrombin-like enzyme in the same amount was measured by the change of absorbance at 405 nm. Both recombinant protein and native protein showed the similar pattern in the catalytic activity of thrombin-like enzyme on several synthetic chromogenic substrates, and recombinant thrombin-like enzyme demonstrated higher specific activity (See FIG. 1a). To identify actual blood hemostatic activity of thrombin-like enzyme, plasma was isolated from human blood and the same amount of recombinant thrombin-like enzyme and native thrombin-like enzyme was added to the plasma according to concentration, and then PT (prothrombin time) was measured by ACL automated blood coagulation instrument. The result demonstrated that addition of thrombin-like enzyme increase the clotting time in a concentration-dependent manner and the specific activity of recombinant thrombin-like enzyme was 1.5 times greater than native enzyme like the result of activity measurement using synthetic chromogenic substrate (See Fig. 1b).

### EXAMPLE 6

Fibrin Clotting Activity of Recombinant Thrombin-like Enzyme

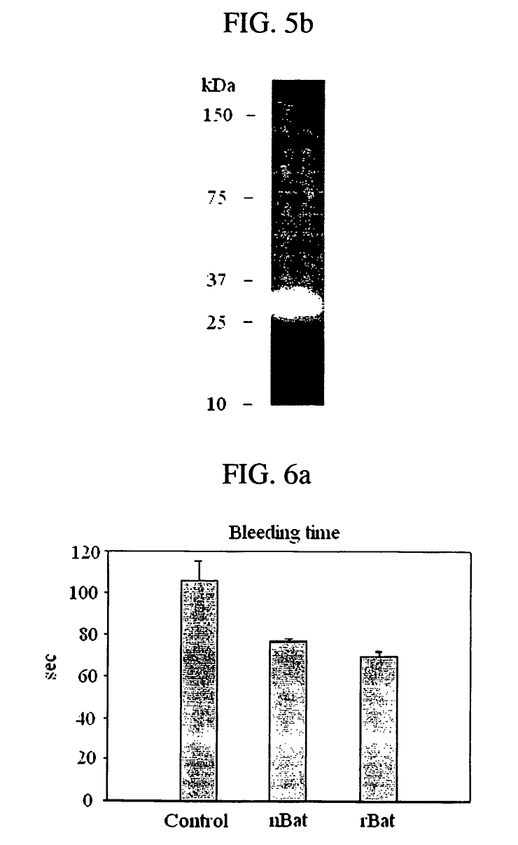

In vitro fibrin clotting activity test and reverse zymography test were performed with recombinant thrombin-like enzyme and native thrombin-like enzyme. As shown in FIG,. 5a, addition of recombinant thrombin-like enzyme to 0.5% human fibrinogen solution made insoluble fibrin clot, which was isolated from water-soluble dye. As shown in FIG. 5b of reverse zymography, insoluble fibrin clot was formed at the location of recombinant thrombin-like enzyme on 0.5% fibrinogen-agar plate.

### EXAMPLE 7

### Reduction of Bleeding Time and Whole Blood Coagulation Time by Recombinant Thrombin-like Enzyme in Animal Experimental Model

To check the clinical application possibility of recombinant thrombin-like enzyme, bleeding time reduction was compared with native thrombin-like enzyme in rat model. The about 1 ml sample (1 NIH unit/kg), which is the similar amount like that used in clinical application, was injected on the tail vein of 8-week-old rats. After 1 and half hours, the tail of rats was transected at 5 mm from the tip and the time to stop bleeding was measured with the tail in PBS. The control was injected with only PBS. The each group had 5 animals.

As shown in FIG. 6a, the bleeding time of animals injected with recombinant thrombin-like enzyme or native thrombin-like enzyme was shorter than the control, which was the result of hemostatic activity of thrombin-like enzyme. The hemostatic activity of recombinant thrombin-like enzyme was better than the native.

In addition, whole blood coagulation time reduction in treated animal model was measured. Recombinant thrombin-like enzyme and native thrombin-like enzyme (2 NIH unit/kg, respectively) were injected on the tail vein of rats. After 1 hour and 4 hours, respectively, the blood was collected and the whole blood coagulation time was measured. The control was injected with only PBS. Each group had 5 animals. The whole blood coagulation time was measured as follows. The collected blood 0.5 ml was mixed 10 mM CaCl₂ in 1.5 ml eppendorf tube, and then the blood coagulation time was measured with agitation of 2 rpm/min and 45° incline. The whole blood coagulation time of blood treated with thrombin-like enzymes was shorter than the control like the result of bleeding time test, and the activity of recombinant thrombin-like enzyme was stronger than the activity of native thrombin-like enzyme (See FIG. 6b).

### EXAMPLE 8

### Changes of PT, APTT and TT by Recombinant Thrombin-like Enzyme in Animal Experimental Model

As described above, several experiments demonstrated that recombinant thrombin-like enzyme has the hemostatic activity to form insoluble fibrin clot like native thrombin-like enzyme. Thus, to investigate whether this recombinant thrombin-like enzyme has an influence on other blood coagulation factor, the PT, APTT and TT changes of blood was measured in animal experimental model. Recombinant thrombin-like enzyme and native thrombin-like enzyme (0.1 NIH unit/kg, respectively) were injected on the tail vein of rats. After 2 hours, the blood was collected and the plasma was isolated. The changes of PT (prothrombin time), APTT (activated partial thromboplastin time) and TT (thrombin time) for each isolated plasma were measured by using automated ACL blood clotting tester. This changes is a in vivo assay method to indirectly investigate the changes of several coagulation factors in mammalian blood coagulation system. The control animals were injected with only PBS and each group had 5 animals. All results were similar, and plasmas of animals injected with the sample showed very slight increase at each test. However, the difference was not significant (See FIG. 6c). Based on these results, it is believed that recombinant thrombin-like enzyme has no significant influence on other blood coagulation factors compared to the hemostatic activity.

### EXAMPLE 10

### Stability Test of Recombinant Thrombin-like Enzyme and Native Thrombin-like Enzyme

As the results of the above several experiments for activity comparison, it is believed that the activity of recombinant thrombin-like enzyme is stronger than the native, which is expected to relate with the protein stability. Thus, the stability of recombinant thrombin-like enzyme and native thrombin-like enzyme was analyzed by measuring the maintenance of protein activity under several pH conditions. As shown in FIG. 7, recombinant thrombin-like enzyme was more stable under each condition. This result is expected to relate with the purity of finally isolated and purified protein, and it was identified by the above SDS-PAGE analysis that the purity of recombinant thrombin-like enzyme was higher than the native.

Acute Toxicity Test

To investigate the acute toxicity of one intravenous administration of recombinant thrombin-like enzyme that can be used as the hemostatic agent or therapeutic and preventing agent of thrombosis, recombinant thrombin-like enzyme was intravenously injected to rat. The dead number was measured for 7 days after administration to determine minimum lethal dose (MLD). The result showed that the MLD of male rats was over 600 NIH unit/kg and the MLD of female rats was 600 NIH unit/kg. These figures were very decreased when considering that the LD50 of native thrombin-like enzyme is about 300 NIH unit/kg. Therefore, within the range of effective amount indicated below, it is expected that the hemostatic agent or therapeutic and preventing agent of thrombosis comprising the recombinant thrombin-like enzyme of the present invention as effective agent is adequately safe.

As described in detail and proven above, the present invention provides yeast-expressed recombinant thrombin-like enzyme, mass production process of transformed yeast, preparation method and purification method of recombinant thrombin-like enzyme, and hemostatic agent or therapeutic and preventing agent for thrombosis comprising the enzyme as effective agent. Recombinant thrombin-like enzyme can be mass-produced because it is purified through two steps of hydrophobic column and affinity column from a large amount of culture fluid of transformed yeast. According to the present invention, recombinant thrombin-like enzyme has effective hemostatic effect and no significant influence on other blood coagulation factors, and the minimal lethal dose of recombinant thrombin-like enzyme in acute toxicity test is very low compared to the native protein. Therefore, the hemostatic agent comprising recombinant thrombin-like enzyme as effective agent will have effective availability in clinical field without side effects and toxicity compared to the therapy using the native protein.

### INDUSTRIAL APPLICABILITY

As above, according to the present invention, recombinant thrombin-like enzyme of the present invention effectively decrease bleeding time and blood coagulation time and does not have effects on other blood coagulation factors, therefore can be used as hemostatic agent. In addition, the toxicity of recombinant thrombin-like enzyme is shown in acute toxicity test to be low compared to native thrombin-like enzyme. Thus the hemostatic agent or therapeutic and preventing agent for thrombosis comprising the recombinant thrombin-like enzyme of the present invention as effective agent can be used availably and effectively without side effects and toxicity compared to the existing therapy using native thrombin-like enzyme.

The present invention has been described in detail. However, it should be understood that the detailed description and specific examples, while indicating preferred embodiments of the invention, are given by way of illustration only, since various changes and modifications within the spirit and scope of the invention will become apparent to those skilled in the art from this detailed description.

What is claimed is:

1. Yeast expression vector pPIC-rBAT comprising recombinant thrombin-like enzyme.

2. The expression vector according to claim 1, wherein the recombinant thrombin-like enzyme is batroxobin protein listed in Sequence NO: 1.

3. Yeast (Pichia pastoris) GS115 transformed with the expression vector pPIC-rBAT of claim 1.

4. Preparation method of thrombin-like enzyme, comprising culture and fermentation of the transformed microorganism of claim 3 and process to obtain recombinant thrombin-like enzyme therefrom.

5. The preparation method according to claim 4, wherein the method is to incubate the transformed microorganism in the medium of pH 5.0-7.0 at 20-40° C. for 12-14 hours and then incubate the cells obtained by centrifugation of culture in the medium of pH 5.0-7.0 containing 0.5-1.5% (v/v) methanol at 20-40° C. for 72-120 hours.

6. Preparation method of recombinant thrombin-like enzyme, comprising
a) step to obtain active fraction of recombinant thrombin-like enzyme from the expressed culture of claim 4 or claim 5 by using hydrophobic chromatograpy; and
b) step to purify recombinant thrombin-like enzyme by applying the active fraction to affinity chromatography.


SEQUENCE LISTING (3 sequences)

SEQ ID NO: 1 - protein, 231 aa, Bothrops atrox moojeni
VIGGDECDINEHPFLAFMYYSPRYFCGMTLINQEWVLTAAHCNRRFMRIHLGKHAGSVANYDEVVRYPKEKFICPNKKKNVITDKDIMLIRLDRPVKNSEHIAPLSLPSNPPSVGSVCRIMGWGAITTSEDTYPDVPHCANINLFNNTVCREAYNGLPAKTLCAGVLQGGIDTCGGDSGGPLICNGQFQGILSWGSDPCAEPRKPAFYTKVFDYLPWIQSIIAGNKTATCP

SEQ ID NO: 2 - DNA, 28 bp, Artificial Sequence
  N-terminal primer
ctcgagaaaagagtcattggaggtgatg

SEQ ID NO: 3 - DNA, 41 bp, Artificial Sequence
  C-terminal primer
ttcacgggcaagtcgcagttttatttcctgcaataatcgtc